(19) United States
(12) Patent Application Publication
(10) Pub. No.: US 2018/0185838 A1
(43) Pub. Date: Jul. 5, 2018

(54) A BIOCHEMICAL ANALYTICAL TECHNIQUE

(71) Applicant: SIEMENS AKTIENGESELLSCHAFT, München (DE)

(72) Inventors: Ralph Grothmann, München (DE); Walter Gumbrecht, Herzogenaurach (DE); Mark Matzas, Heroldsbach (DE); Peter Paulicka, Röttenbach (DE); Stefanie Vogl, Konzell (DE); Hans-Georg Zimmermann, Starnberg/Percha (DE)

(21) Appl. No.: 15/740,431

(22) PCT Filed: Jul. 2, 2015
(86) PCT No.: PCT/EP2015/065097

§ 371 Date: Dec. 28, 2017

(51) Int. Cl. B01L 3/00; G01N 33/487; G06F 19/24; G01N 33/493; G01N 33/49; G01N 33/53

(52) U.S. Cl. CPC B01L 3/5027; G01N 33/48785; G06F 19/24; G01N 33/493; B01L 2300/0663; G01N 33/5308; B01L 2200/143; B01L 2300/024; G01N 33/492

(57) ABSTRACT

A biochemical analytical device and a biochemical analytical method for determining an analyte in a test sample are provided. In the technique, the biochemical analytical device includes a sample port to receive the test sample, a sensor to probe the test sample and to generate sensor data, and a processor. The sensor data corresponds to the analyte in the test sample. The processor receives the sensor data from the sensor and selects a non-linear function for the received sensor data. The processor fits the selected non-linear function to the sensor data. Additionally, the processor compares the fitted non-linear function to a reference data to determine the analyte in the test sample.

The present patent document is a § 371 nationalization of PCT Application Serial Number PCT/EP2015/065097, filed Jul. 2, 2015, designating the United States, which is hereby incorporated by reference.

### TECHNICAL FIELD

The present disclosure is related to biochemical analysis and more particularly to biochemical analysis devices and biochemical analysis methods for determining an analyte in a test sample.

### BACKGROUND

Modern day medical and clinical sciences rely substantially on biochemical assay techniques. A biochemical assay is an analytic procedure in laboratory medicine, pharmacology, environmental biology, continuous delivery, and molecular biology for qualitatively assessing or quantitatively measuring a presence or an amount or a functional activity of a target entity. The target entity may be a drug or a biochemical substance or a cell, for example, microbial cells in an organism or organic sample. The target entity may be referred to as an analyte, test entity, or target of the assay. The assay may aim to measure an intensive property of the analyte and express it in the relevant measurement unit for, e.g., molarity, concentration, density, functional activity, degree of some effect in comparison to a standard, etc.

Present day biochemical assays are performed by biochemical techniques that involve biochemical analysis devices having sensors or biosensors and use biochemical analysis methods. An example of a biochemical analysis device is a lab-on-a-chip device. The biochemical analysis devices may have one or more sensors, for example, electrochemical sensors which may be arranged in columns and rows. The sensors detect the presence of specific analytes, for example, in some biochemical analysis devices. The sensors are coated with molecules to which the analyte to be detected binds specifically, whereas in some other sensors there may be a chemical entity with which the analyte reacts directly or indirectly. The specific binding or the specific reaction is detected electrochemically by changes in current and/or voltage. In this way, biochemical substances, (e.g., toxins, microbial load, chemical entities, antibodies, peptides, or DNA), may be detected in solutions to be examined, (e.g., blood or urine).

Subsequently, the measured electrochemical signals from the sensors, e.g., sensor data may be processed directly by integrated circuits in the biochemical analysis device or may be read out from the biochemical analysis device by an external evaluation unit. The analysis of the measured electrochemical signal over time duration for which the analysis is performed is of utmost importance to get accurate and robust results from the biochemical analysis technique.

### SUMMARY AND DESCRIPTION

The object of the present disclosure is to provide a biochemical technique, a method and a device, for determining an analyte in a test sample. It is desired that the technique is sensitive, reliable and robust.

The scope of the present disclosure is defined solely by the appended claims and is not affected to any degree by the statements within this summary. The present embodiments may obviate one or more of the drawbacks or limitations in the related art.

The above objects are achieved by a biochemical analytical device for determining an analyte in a test sample and by a biochemical analytical method for determining an analyte in a test sample.

According to an aspect of the present disclosure, a biochemical analytical device for determining an analyte in a test sample is presented. The biochemical analytical device, hereinafter referred to as the device, includes a sample port, at least a sensor, and a processor. The sample port receives the test sample to be analyzed. The sensor analyzes or probes the test sample and generates sensor data. The sensor data corresponds to the analyte in the test sample. The processor receives the sensor data from the sensor, selects a non-linear function for the received sensor data, fits the selected non-linear function to the sensor data, and compares the fitted non-linear function to a reference data to determine the analyte in the test sample. As a result of the fitting non-linear function to the sensor data, a substantial number of data points from the sensor data overlap or fit the non-linear function, and thus, when the fitted non-linear function is compared to the reference data, the results obtained represent the substantial number of data points from the sensor data. This provides sensitive, reliable, and robust results.

In an embodiment, the biochemical analytical device is a lab-on-a-chip device. This provides an advantageous embodiment of the biochemical analytical device because of the portability, compactness, ease of use, and faster analysis and response times of the lab-on-a-chip device.

In another embodiment of the biochemical analytical device, the non-linear function is a parametric fit function. Thus, the processor determines parameters that may be used further to compare with the reference data in form of simple reference table, such as a look up table, to determine the analyte.

In another embodiment of the biochemical analytical device, the parametric fit function is a logistic function. The logistic function is a simple and robust fitting function that provides sensitivity of the biochemical analytical device.

In another embodiment of the biochemical analytical device, the parametric fit function is a hyperbolic tangent function. The hyperbolic tangent function is a simple and robust fitting function that further provides the sensitivity of the biochemical analytical device.

In another embodiment of the biochemical analytical device, the processor determines a steepest ascent of the fitted non-linear function. The steepest ascent of the fitted non-linear function, along with a position of the steepest accent on the non-linear fit, is indicative of the type of analyte. Thus, the device is capable of determining the absence or presence of different types of analytes. Furthermore, the steepest ascent of the fitted non-linear function, along with the position of the steepest accent on the non-linear fit, may also provide indication on quantitative measurement of the analyte.

In another embodiment of the biochemical analytical device, the processor determines a time of occurrence of the steepest ascent. The time of occurrence of the steepest ascent of the fitted non-linear function, along with maximum value of the fitted non-linear function at the steepest ascent, is indicative of quantitative measurement of the analyte. Thus, the device is capable of quantitative determination of the analyte. Furthermore, the time of occurrence of the steepest ascent of the fitted non-linear function, along with maximum value of the fitted non-linear function at the steepest ascent, may also provide indication on the type of analyte and thus help resolution between different types of analytes.

According to another aspect of the present technique, a biochemical analytical method for determining an analyte in a test sample is presented. In the biochemical analysis method, hereinafter referred to as the method, the test sample is analyzed with a sensor of a biochemical analytical device to generate sensor data. The sensor data generated corresponds to the analyte in the analyzed test sample. Subsequently, in the method, the sensor data from the sensor is received by a processor. Thereinafter, a non-linear function is selected by the processor for the received sensor data. Subsequently, the selected non-linear function is fitted by the processor to the sensor data. Additionally, in the method, the fitted non-linear function is compared by the processor to a reference data to determine the analyte in the test sample.

In an embodiment of the method, the biochemical analytical device is a lab-on-a-chip device. This provides an advantageous embodiment of the method wherein the method is implemented with the lab-on-a-chip device that is portable, easy to use, and provides faster analysis and response times for the method.

In another embodiment of the method, the non-linear function is a parametric fit function. Thus, in the method, parameters are determined that may be used further to compare with the reference data in form of simple reference table, such as a look up table, to determine the analyte.

In another embodiment of the method, the parametric fit function is a logistic function. The logistic function is a simple and robust fitting function that provides sensitivity of the method.

In another embodiment of the method, the parametric fit function is a hyperbolic tangent function. The hyperbolic tangent function is a simple and robust fitting function that further provides the sensitivity of the biochemical analytical method.

In another embodiment of the method, in comparing, by the processor, the fitted non-linear function to the reference data, a steepest ascent of the fitted non-linear function is determined by the processor. The steepest ascent of the fitted non-linear function, along with a position of the steepest accent on the non-linear fit, is indicative of the type of analyte. Thus, the method is capable of determining the absence or presence of different types of analytes. Furthermore, the steepest ascent of the fitted non-linear function, along with the position of the steepest accent on the non-linear fit, may also provide indication on quantitative measurement of the analyte.

In another embodiment of the method, in comparing, by the processor, the fitted non-linear function to the reference data, a time of occurrence of the steepest ascent is determined by the processor. The time of occurrence of the steepest ascent of the fitted non-linear function, along with maximum value of the fitted non-linear function at the steepest ascent, is indicative of quantitative measurement of the analyte. Thus, the method is capable of quantitative determination of the analyte. Furthermore, the time of occurrence of the steepest ascent of the fitted non-linear function, along with maximum value of the fitted non-linear function at the steepest ascent, may also provide indication on the type of analyte and thus help resolution between different types of analytes.

### BRIEF DESCRIPTION OF THE DRAWINGS

The present technique is further described hereinafter with reference to illustrated embodiments shown in the accompanying drawing, in which:

FIG. 1 schematically illustrates an example of a biochemical analytical device for determining an analyte in a test sample.

FIG. 2 illustrates a flow chart representing an example of a biochemical analytical method for determining the analyte in the test sample.

FIG. 3 illustrates exemplary curves used in the method for determining the analyte in the test sample, in accordance with aspects of the present disclosure.

### DETAILED DESCRIPTION

Hereinafter, above-mentioned and other features of the present disclosure are described in detail. Various embodiments are described with reference to the drawings, wherein like reference numerals are used to refer to like elements throughout. In the following description, for purpose of explanation, numerous specific details are set forth in order to provide a thorough understanding of one or more embodiments. It may be noted that the illustrated embodiments are intended to explain, and not to limit the disclosure. It may be evident that such embodiments may be practiced without these specific details.

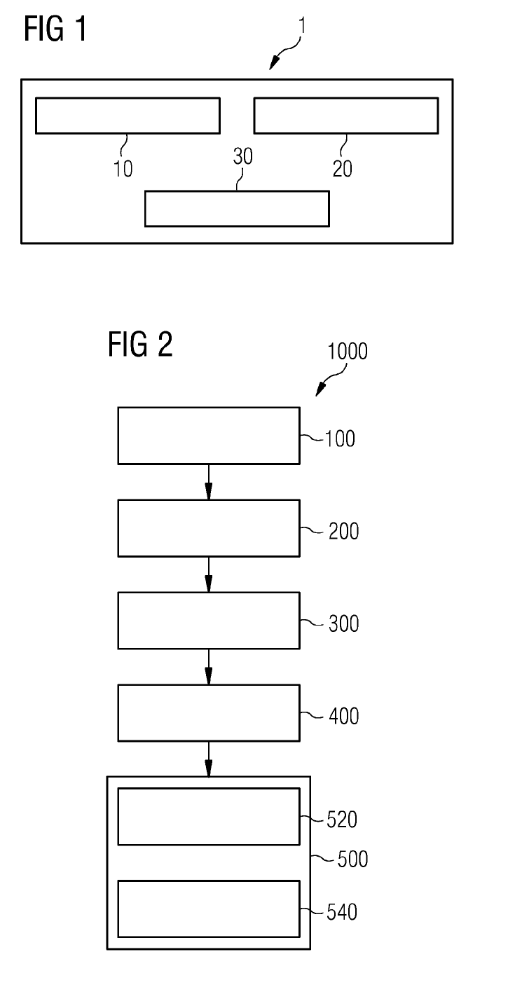

As depicted in FIG. 1, according to an aspect of the present disclosure, a biochemical analytical device 1, (hereinafter referred to as the device 1), for determining an analyte in a test sample is presented. The device 1 includes a sample port 10, at least one sensor 20, and a processor 30. The sample port 10 receives the test sample to be analyzed. The sensor 20 analyzes or probes or investigates the test sample and generates sensor data. The sensor data corresponds to the analyte in the test sample. The processor 30 receives the sensor data from the sensor 20. Furthermore, the processor 30 selects a non-linear function for the received sensor data and then fits the selected non-linear function to the sensor data. The processor 30 compares the fitted non-linear function to a reference data to determine the analyte in the test sample. The working of the device 1 and different embodiments have been explained further using FIG. 1 in combination with FIG. 2 which illustrates a flow chart representing a biochemical analytical method 1000, (hereinafter referred to as the method 1000), for determining the analyte in the test sample, in accordance with aspects of the present technique.

As used herein, the term “analyte” is a substance or chemical constituent that is of interest in the biochemical analytical method or that may be detected by the sensor 20 of the biochemical analytical device 1 and includes, but is not limited to, a drug, a cell of the host or a foreign cell such as a microbial cell, (e.g., bacteria, virus, etc.), a toxin, byproducts of a host cell or of a foreign cell, allergens, products or byproducts of metabolic or enzymatic processes, chemical compounds, and so on and so forth.

For the purposes of the present technique, the phrase “determining an analyte in the test sample” or like phrases, as used herein, refers to probing, checking, evaluating, testing, scrutinizing, or examining the test sample for presence or absence of the analyte in the test sample, and may optionally include quantifying the analyte present in the test sample.

The device 1 may be any biochemistry analyzer such as a lab-on-a-chip device. In biosensor technology, lab-on-a-chip systems are used in order to be able to carry out biochemical analyses in parallel and thus several analytes of different type may be determined simultaneously by the present technique. The lab-on-a-chip device is a microfluidic arrangement or instrument and includes a chip having an array of sensors 20 integrated on a support, which may include a plastic card. The array of sensors 20 includes, for example, electrochemical sensors 20 arranged in columns and rows on the chip. The test sample, (e.g., blood or urine), is placed on or in the sample port 10. In the device 1, the sensors 20 are coated with molecules, to which the analyte, (e.g., the substances or entity to be detected), binds specifically. Different sensors may be coated with different molecules having specific binding affinity for different types of analytes. Whereas, in some other sensors 20, there may be a chemical entity, (e.g., a coating of a specific chemical compound), with which the analyte reacts directly or indirectly. The specific binding of the analyte and the molecules on the sensor 20 and/or the specific reaction of the analyte and the sensor 20 are detected electrochemically and manifested or detected by changes in current and/or voltage delivered as an output of the sensor 20 as form of the sensor data. In this way, the analytes are detected by analyzing the test samples, (e.g., blood or urine). In the method 1000, in act 100, the test sample is analyzed with the sensor 20 of the device 1 to the generate sensor data. The generated sensor data corresponds to the analyte in the analyzed test sample in act 100. The sensor data correspond to or represents the type of analyte and/or the quantity of analyte present in the test sample.

Subsequently, in the method 1000, as well as in the working of the device 1, the measured electrochemical signals provided by the sensor 20 in form of the sensor data is received by the processor 30 in act 200. In one embodiment of the device 1, the processor 30 is physically an integral part of the device 1. For example, the processor 30 may be present as an integrated circuit 30 in the lab-on-a-chip device 1 embedded within the support or chip of the device 1, whereas in an alternate embodiment of the device 1, the processor 30 may be present as a separate physical entity for example a biochemistry sensor array device connected to an external processing unit.

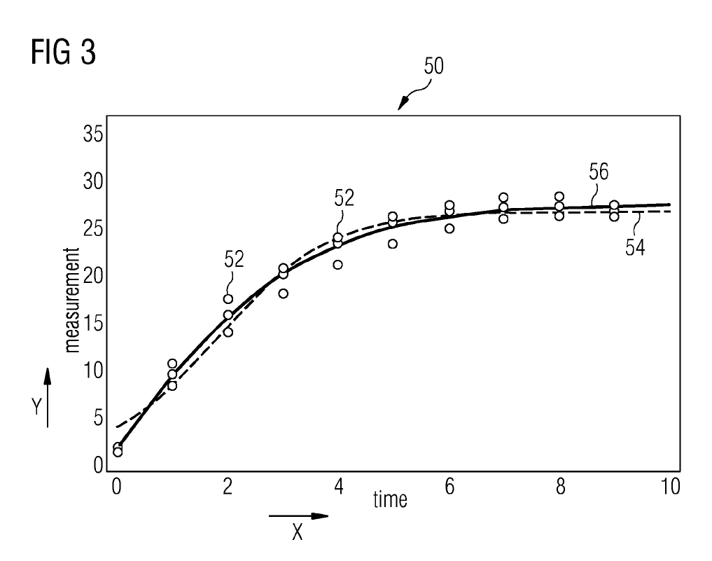

In the device 1, as well as in the method 1000, in act 300, a non-linear function is selected by the processor 30 for the received sensor data by the processor 30. In an embodiment of the method 1000, the non-linear function is a parametric fit function. In an embodiment of the device 1, the processor 30 is configured to select a parametric fit function. The parametric fit function may be, but not limited to a, logistic function with parameters or a hyperbolic tangent function with parameters. The method 1000, as well as in the device 1, wherein the parametric fit function is the logistic function with parameters or the hyperbolic tangent function with parameters have been explained later with reference to FIG. 3.

Subsequent to act 300, in the method 1000, in act 400, the selected non-linear function is fitted to the sensor data. The fitting of the selected non-linear function is performed by the processor 30. The fitting, or also referred to as curve fitting, is a process of constructing a curve, or mathematical function, that has the best fit to a series of data points, possibly subject to constraints. In the present technique, data points are points 52 (shown in FIG. 3) that form the sensor data. In act 400 of curve fitting, the selected non-linear function is fitted or matched to the points 52 (as shown in FIG. 3) such that the selected non-linear function passes through or considers a substantial number of the points 52. The technique of curve fitting is highly known and pervasively used in the field of statistical analysis and thus has not been explained herein in details for sake of brevity.

In the method 1000, after act 400, in act 500, the fitted non-linear function or the fitted non-linear curve is compared to a reference data to determine the analyte in the test sample. In one embodiment, the comparison is performed directly between a shape of the fitted non-linear function obtained as a result of act 400 and the reference data, which is represented as different non-linear curves and the correlation of different shapes of non-linear curves with different types of analytes and their respective concentrations. In another embodiment, the parameters of the fitted non-linear function are used to compare with the reference data.

As used herein, the term “reference data” refers to a collection of data representing relation between different characteristics of curves or data sets resulting from fitted non-linear functions, such as shape of the curve, location of a maxima on the curve, and/or value of the maxima, etc., with presence or absence of different analytes and/or the quantitative indication such as concentration of different analytes. An example of reference data is a look up table that represents the correlation between different types of analytes with the time of occurrence of maxima, shape of the curve, or value of the maxima. The look up table may also include the relation of different types of analytes and their concentrations with the characteristics of the curve. Another example of reference data is, but not limited to, a standard curve representing relation between different analyte concentrations and related rate of change in sensor data. The method of using and creating such reference data, (also sometimes referred to as standard curves), result look up table, or reference curves, is a well known and pervasively used standard laboratory technique and thus has not been described herein for sake of brevity.

In an embodiment of the device 1, the processor 30 is configured to determine a steepest ascent of the fitted non-linear function and/or to determine a time of occurrence of the steepest ascent. The steepest ascent may also be understood as the steepest climb or rise in the curve leading to maxima on the curve. In a related embodiment of the method 1000, within act 500, the steepest ascent of the fitted non-linear function is determined in act 520 by the processor 30. Furthermore, in another related embodiment of the method 1000, within act 500, the time of occurrence of the steepest ascent is determined in act 540 by the processor 30.

Now referring to FIG. 3, in an exemplary graph 50 use of the logistic function and the hyperbolic tangential function as the selected non-linear function or the non-linear parametric fit function fitted to the sensor data is depicted. In the graph of FIG. 3, time is depicted on ‘X’ axis and may be measured in unit of time for example seconds. When the sensor 20 of the device 1 is engaged with the test sample, (e.g., when the sensor 20 is turned on to probe the test sample), the recording of time starts, and along with it the measurements from the sensor 20 are recorded along the ‘Y’ axis, as shown in FIG. 3, and the measurements may be made or recorded in unit of electrical voltage or electric current intensity as sensed by the sensor as a result of probing the test sample and interacting with the analyte. The measurements are made over time along the ‘X’ axis and recorded as data points 52. The multiple data points at the same time instance as shown in FIG. 3 may be due to repeated measurements in different run cycles or may be due to measurements made by different sensors 20 at the same time instance. The entire collection of all such measurements or the data points 52 is referred to as the sensor data and is received by the processor 30.

### A. Use of the Logistic Function

The processor 30 after receiving the sensor data fits the logistic function with parameters to the sensor data. The logistic function selected by the processor 30 and fitted subsequently may be represented by the following equation:

$$f ( x ) = \frac{a}{b + {e}^{( - 4 cx )}} \qquad ( i )$$

wherein, f(x) denotes the logistic function, e denotes the exponential, and a, b, c are the parameters.

The fitted logistic function is represented by an exemplary first curve 54 in the graph 50. As is clear from the exemplary representation depicted in FIG. 3, the first curve 54 considers or passes through or overlaps with a substantial number of the data points 52 and represents the sensor data in its entirety and is better than a linear fit, and thus when compared to the reference data or when used to draw inference about the analyte in the test sample the first curve 54 delivers a more accurate and sensitive result having considered the substantial number of the data points 52 from the sensor data.

Moreover, as mentioned earlier, the steepest ascent of the fitted logistic function is determined by the processor 30, wherein the steepest ascent is represented by the following equation:

$${x}_{max} = - \frac{log b}{4 c} \qquad ( ii )$$

when the parametric fit function is the logistic function, and wherein xₘₐₓ represents the steepest ascent or a maxima on the first curve 54, and b, c represent the parameters from the equation (i) of the logistic function presented earlier.

Furthermore, the time of occurrence of the steepest ascent, e.g., a time value along the X axis in graph 50 is also determined by the processor 30. The time of occurrence of the steepest ascent is represented by the following equation:

$${{f}^{′}}_{max} ( x ) = \frac{ac}{b} \qquad ( iii )$$

when the parametric fit function is the logistic function and wherein f′ₘₐₓ(x) denotes the time along the X axis in graph 50, e.g., the time of occurrence of the steepest ascent xₘₐₓ as denoted in equation (ii) of the fitted logistic first curve 54. As may be seen from the equations (ii) and (iii), the determination of the steepest ascent and the time of occurrence of the steepest ascent are simple as they are performed easily by using simple equations and using the parameters a, b, c from equation (i).

### B. Use of the Hyperbolic Tangent Function

The processor 30, after receiving the sensor data, fits the hyperbolic tangent function with parameters to the sensor data. The hyperbolic tangent function selected by the processor 30 and subsequently fitted may be represented by the following equation:

f(x)=a tan h(b+cx) (iv)

wherein f(x) denotes the hyperbolic tangent function and a, b, c are the parameters.

The fitted hyperbolic tangent function is represented by an exemplary second curve 56 in the graph 50. As is clear from the exemplary representation depicted in FIG. 3, the second curve 56 considers or passes through or overlaps with a substantial number of the data points 52 and represents the sensor data in its entirety and better than a linear fit, and thus when compared to the reference data or when used to draw inference about the analyte in the test sample the second curve 56 delivers a more accurate and sensitive result having considered the substantial number of the data points 52 from the sensor data.

Moreover, as mentioned earlier, the steepest ascent of the fitted hyperbolic tangent function is determined by the processor 30, wherein the steepest ascent is represented by the following equation:

$${x}_{max} = - \frac{ b}{c} \qquad ( v )$$

when the parametric fit function is the hyperbolic tangent function, and wherein xₘₐₓ represents the steepest ascent or a maxima on the second curve 56 and b, c represent the parameters from the equation (iv) of the hyperbolic tangent function presented earlier.

Furthermore, the time of occurrence of the steepest ascent, e.g., a time value along the X axis in graph 50 is also determined by the processor 30. The time of occurrence of the steepest ascent is represented by the following equation:

f′ₘₐₓ(x)=ac (vi)

when the parametric fit function is the hyperbolic tangent function and wherein f′ₘₐₓ(x) denotes the time along the X axis in graph 50, e.g., the time of occurrence of the steepest ascent xₘₐₓ as denoted in equation (v) of the fitted hyperbolic tangent second curve 56. As may be seen from the equations (v) and (vi), the determination of the steepest ascent and the time of occurrence of the steepest ascent are simple as they are performed by using simple equations and using the parameters a, b, c from equation (iv).

While the present technique has been described in detail with reference to certain embodiments, it should be appreciated that the present technique is not limited to those precise embodiments. Rather, in view of the present disclosure which describes exemplary modes for practicing the disclosure, many modifications and variations would present themselves, to those skilled in the art without departing from the scope and spirit of the disclosure. The scope of the disclosure is, therefore, indicated by the following claims rather than by the foregoing description. All changes, modifications, and variations coming within the meaning and range of equivalency of the claims are to be considered within their scope.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

1. A biochemical analytical device configured to determine an analyte in a test sample, the biochemical analytical device comprising:
a sample port configured to receive the test sample;
a sensor configured to analyze the test sample and to generate sensor data corresponding to the analyte in the analyzed test sample; and
a processor (30) configured to:
receive the sensor data from the sensor,
select a non-linear function for the received sensor data,
fit the selected non-linear function to the sensor data, and
compare the fitted non-linear function to a reference data to determine the analyte in the test sample.

2. The biochemical analytical device of claim 1, wherein the biochemical analytical device is a lab-on-a-chip device.

3. The biochemical analytical device of claim 1, wherein the non-linear function is a parametric fit function.

4. The biochemical analytical device of claim 3, wherein the parametric fit function is a logistic function.

5. The biochemical analytical device of claim 3, wherein the parametric fit function is a hyperbolic tangent function.

6. The biochemical analytical device of claim 1, wherein the processor is further configured to determine a steepest ascent of the fitted non-linear function.

7. The biochemical analytical device of claim 6, wherein the processor is further configured to determine a time of occurrence of the steepest ascent.

8. A biochemical analytical method to determine an analyte in a test sample, the biochemical analysis method comprising:
analyzing the test sample with a sensor of a biochemical analytical device;
generating sensor data from the analyzed test sample;
receiving, by a processor, the sensor data from the sensor;
selecting, by the processor, a non-linear function for the received sensor data;
fitting, by the processor, the selected non-linear function to the sensor data; and
comparing, by the processor, the fitted non-linear function to a reference data to determine the analyte in the test sample.

9. The biochemical analytical method of claim 8, wherein the biochemical analytical device is a lab-on-a-chip device.

10. The biochemical analytical method claim 8, wherein the non-linear function is a parametric fit function.

11. The biochemical analytical method of claim 10, wherein the parametric fit function is a logistic function.

12. The biochemical analytical method of claim 10, wherein the parametric fit function is a hyperbolic tangent function.

13. The biochemical analytical method of claim 8, wherein in the comparing, a steepest ascent of the fitted non-linear function is determined by the processor.

14. The biochemical analytical method of claim 13, wherein in the comparing, a time of occurrence of the steepest ascent is determined by the processor.

15. The biochemical analytical method of claim 9, wherein in the comparing, a steepest ascent of the fitted non-linear function is determined by the processor.

16. The biochemical analytical method of claim 15, wherein in the comparing, a time of occurrence of the steepest ascent is determined by the processor.

17. The biochemical analytical device of claim 2, wherein the processor is further configured to determine a steepest ascent of the fitted non-linear function.

18. The biochemical analytical device of claim 17, wherein the processor is further configured to determine a time of occurrence of the steepest ascent.
